(19) United States
(12) Patent Application Publication
(10) Pub. No.: US 2021/0022679 A1
(43) Pub. Date: Jan. 28, 2021

(54) Sensor Devices and Methods for Detecting when a Sensor Device is Worn

(71) Applicant: SENS INNOVATION APS, Copenhagen N (DK)

(72) Inventors: Kasper Lykkegaard, Copenhagen S (DK); Morten Kjærgaard, Frederiksberg C (DK)

(21) Appl. No.: 17/000,282

(22) Filed: Aug. 22, 2020

Related U.S. Application Data
(63) Continuation of application No. PCT/DK2019/050053, filed on Feb. 19, 2019.

(30) Foreign Application Priority Data
Feb. 23, 2018 (DK) PA 2018 00089
Jan. 31, 2019 (DK) PA 2019 00147

(51) Int. Cl. A61B 5/00; A61B 5/11; A61B 5/0205; A61B 5/103; A61B 5/053; G16H 40/67; H04L 29/06

(52) U.S. Cl. CPC A61B 5/6843; A61B 5/024; A61B 5/1116; A61B 5/0205; A61B 5/1032; A61B 5/0531; A61B 5/6835; A61B 5/6833; G16H 40/67; H04L 63/0428; A61B 2562/0257; A61B 2562/0219; A61B 2562/0271; A61B 2562/08; A61B 5/0002

(57) ABSTRACT

A sensor device (2) is disclosed. The sensor device (2) comprises a processor (14), a memory (24), an antenna (6), a power source (e.g. a battery), one or more sensing elements (12) configured to provide measurement data and store the measurement data in the memory (24) and a communication unit (16) configured to transfer data from the one or more sensing elements (12) and data stored in the memory to a remote unit (28). The sensor device (2) comprises a contact detection unit configured to detect if the sensor device (2) is in contact with or in close proximity to the skin (80) of a user (32, 32′) if at least two of the at least three measurements made by different sensing elements (12) indicate that the sensor device (2) is in contact with or in close proximity to the skin (80) of a user (32, 32′).

### CROSS-REFERENCE TO RELATED APPLICATIONS

This application is a continuation under 35 U.S.C. 111 of International Patent Application No. PCT/DK2019/050053, filed Feb. 19, 2019, which claims the benefit of and priority to Danish Application Nos. PA 2018 00089, filed Feb. 23, 2018, and PA 2019 00147, filed Jan. 31, 2019, each of which is hereby incorporated by reference in its entirety.

### FIELD OF INVENTION

The present invention relates to devices and methods for detecting when a sensor device is worn.

### BACKGROUND

There has been an increasing use of non-invasive wearable sensors for remote health monitoring because the recent decade has introduced cost-effective and easy-to-use systems. Moreover, modern communication and information technologies offer an efficient and cost-effective solution that allows individuals to continue to live in the comfort of their home environment instead of expensive healthcare facilities. At the same time, these systems allow healthcare personnel to monitor important physiological parameters of the patients in real time, assess health conditions, and provide feedback from distant facilities.

These sensors may be adapted to monitor various parameters including temperature, heart rate, acceleration and other physiological parameters using low-cost electronic equipment.

Some sensors will monitor data regardless of whether the sensors are worn or not. This means that some data collected by the sensors is not useful and it introduces the risk of misinterpretation when the data is used for analyses.

Unfortunately, sensors adapted to detect when a sensor is worn often do not provide reliable measurements. Accordingly, there is a risk of detecting useless data and for misinterpretations to occur due to the use of wrong data.

Thus, there is a need for methods and sensors that eliminate the above-mentioned disadvantages of the prior art.

### SUMMARY

It is an object of the invention to provide sensors and methods that can automatically detect when a sensor is worn in a more reliable manner than the prior art.

A sensor device according to the invention is a sensor device comprising:
a processor;
a memory;
an antenna;
a power source (e.g. a battery);
one or more sensing elements configured to provide measurement data and store the measurement data in the memory; and
a communication unit configured to transfer data from the one or more sensing elements and data stored in the memory to a remote unit,
   wherein the sensor device comprises a contact detection unit configured to detect if the sensor device is in contact with or in close proximity to the skin of a user if at least two of the at least three measurements made by different sensing elements indicate that the sensor device is in contact with or in close proximity to the skin of a user.

Hereby, it is possible to provide a sensor that can automatically detect when a sensor is worn in a more reliable manner than prior art sensors.

It may be an advantage that the contact detection unit comprises a capacitive proximity sensor, an accelerometer and a temperature sensor. Hereby, it is possible to apply three different types of measurements to detect if the sensor device is worn.

It may be beneficial that the contact detection unit is configured to apply the capacitive proximity sensor, the accelerometer and the temperature sensor to perform measurements indicating if the sensor device is in contact with or in close proximity to the skin of a user and to determine that the sensor device is in contact with or in close proximity to the skin of a user if at least two of the measurements indicate that the sensor device is in contact with the skin of a user. Hereby, it is possible to provide a reliable way to detect if the sensor device is worn.

In one embodiment, the sensor device is configured to determine that the sensor device is in contact with or in close proximity to the skin of a user if all three measurements indicate that the sensor device is in contact with or in close proximity to the skin of a user. Hereby, it is possible to provide an even more reliable way to detect if the sensor device is worn.

In one embodiment, the sensor device has a unique identity (ID) and a predefined encryption key stored in the sensor device, wherein the sensor device is configured to encrypt the measurement data by using the encryption key before the measurement data is transferred to the remote unit.

Since all data is encrypted, it is possible to provide a sensor device that can safely transmit data using a non-secure communication protocol.

In an embodiment, the sensor device is configured to be attached to the skin of a user or alternatively to be brought in close proximity to the skin of a user. It may be an advantage that the distance between the sensor device and the skin is less than 10 mm. In one embodiment, the sensor device is configured to be arranged in a position in which the distance between the sensor device and the skin is less than 5 mm. In one embodiment, the sensor device is configured to be arranged in a position in which the distance between the sensor device and the skin is less than 2 mm.

The sensor device is configured to wirelessly communicate with the remote unit. Accordingly, the sensor device is configured to wirelessly send encrypted data to the remote unit. This may be done using a non-secure communication protocol because the data is encrypted.

The processor may be any suitable type of processor having any suitable size. It is, however, preferred that a small-sized processor is used in order to limit the overall size of the sensor device. In an embodiment, the sensor device is less than 5 mm thick.

The memory may be any suitable type of memory and may have any required size. In an embodiment, the memory is an electronic solid-state computer storage medium that can be electrically erased and reprogrammed (e.g. a flash memory). A small-sized memory is preferred in order to limit the overall size of the sensor device. The memory may be an integrated part of the processor. In one embodiment, the memory is, however, a separate component from the processor, wherein the memory is electrically connected to the memory.

The communication unit may be any suitable type of communication unit and comprises a transmitting unit configured to wirelessly transmit signals. In one embodiment, the communication unit comprises a receiving unit configured to receive one or more wirelessly sent signals.

The remote unit may be a tablet, a smartphone or another device capable of receiving wireless signals sent from the sensor device.

In one embodiment, the sensor device is configured to encrypt the measurement data and thereafter store the encrypted measurement data in the memory before the encrypted measurement data is transferred (wirelessly transmitted) to the remote unit.

In one embodiment, the sensor device comprises a printed circuit board (PCB) and the processor, the memory, the communication unit and the one or more sensing elements are arranged on the PCB.

Hereby, it is possible to provide a small-sized sensor device. Moreover, it is possible to provide a cost-efficient sensor device.

It may be advantageous that the one or more sensing elements are configured to measure temperature.

In one embodiment, the one or more sensing elements are configured to measure capacitance. In one embodiment, the one or more sensing elements are configured to measure acceleration (such as acceleration in three dimensions).

In one embodiment, the one or more sensing elements are configured to measure heart rate. In one embodiment, the one or more sensing elements are configured to measure changes in skin redness.

In one embodiment, the one or more sensing elements are configured to measure electrical conductivity. Hereby, the sensor device can provide important physiological parameters of patients.

In one embodiment, the one or more sensing elements are configured to measure changes in skin redness and thereby detect the heart rate by counting the subtle changes in skin redness.

It may be an advantage that the sensor device comprises a mounting structure to mount the sensor device in contact with or in close proximity to the skin of a user. Hereby, it is possible to optimize the position of the sensor device and thus provide a number of reliable measurements using the sensor device. In close proximity to the skin could be within a distance less than 10 mm, preferably less than 5 mm such as 0-2 mm.

It may be beneficial that the mounting structure comprises an adhesive portion configured to be detachably attached to the skin of a user. Hereby, it is possible to attach the sensor device in a fast and reliable manner. Moreover, the sensor device can be positioned in various positions, in which the sensor device can provide reliable temperature measurements, electric capacitance measurements and acceleration measurements.

It may be an advantage that the sensor device comprises a break detector arranged and configured to break if the sensor device is detached from a user. Hereby, it is possible to generate an alert in case the sensor device is detached from a user. In one embodiment, the sensor device is configured to automatically generate and transmit one or more alert signals to one or more remote receivers when the break detector breaks.

It is an advantage that the break detector is formed as an electrical wire having a thickness that allows it to break (due to the deformation of the mounting structure, when the sensor device is removed from the skin of a user). Hereby, it is possible to provide a simple, reliable and cost-efficient way of detecting if the sensor device is detached from a user.

In one embodiment, the sensor device comprises a deformable mounting structure and a break detector arranged in a position in which deformation of a deformable mounting structure during detachment of the sensor device causes a structure (e.g. an electrical wire or connector) of the break detector to break.

It may be an advantage that the sensor device comprises a pocket structure configured to receive and contain electronic components of the sensor device. In one embodiment, the sensor device is disposable (designed for single use) and integrated in a patch (plaster).

In one embodiment, the sensor device is configured to estimate the body pose, sleep cycle and sleep time, wherein the estimation is based on accelerometer data collected by the sensor device.

In one embodiment, the sensor device is configured to estimate the sleep cycle on the basis of movements during sleep, their magnitude and time between the movements.

It is well known from literature that a sleep cycle consists of several stages including: awake, N1, N2, N3 and REM. The different stages are characterized by different movement patterns, which can be recognized by the sensor device.

N1: During this short period (lasting several minutes) of relatively light sleep, the heartbeat, respiratory rate, and eye movement activity is decreased, and the muscles relax with occasional twitches. Body movements and active periods, in which the vector magnitude of the measured accelerations is above a predefined threshold T1 are marked and when movements (active periods of a few seconds up to some minutes) are registered with a frequency (within a predefined frequency range), the sleep-cycle N1 is registered.

N2 is a non-REM sleep period of light sleep before one enters deeper sleep. The heart rate and respiratory rate go down, and the muscle activity is decreased even further. The body temperature decreases, and eye movements stop. In this stage, there are fewer movements. When movements (active periods of a few seconds up to some minutes) are registered with a predefined frequency (within a predefined frequency range) the sleep-cycle N2 is registered.

N3 is a longer lasting period of deep sleep. It normally occurs during the first half of the night. The heart rate and respiratory rate decrease to their lowest levels during sleep. The muscles are relaxed, and it is difficult to wake up from this stage of sleep. During this stage we normally only see few movements above the threshold T1 and there are long time periods between the active periods.

REM is the “rapid eye movements” stage, which occurs about 90 minutes after falling asleep. The eyes move rapidly from side to side behind closed eyelids. Mixed frequency brain wave activity becomes closer to that seen in wakefulness. The respiratory rate increases and becomes more irregular (larger dispersion), and the heart rate and blood pressure increase to near waking levels. Most dreaming occurs during REM sleep, although some can also occur in non-REM sleep. The arm and leg muscles become temporarily paralyzed, which prevents you from acting out your dreams. As you age, you sleep less of your time in REM sleep. This is recognized by a period of no movements at all measured (eye-movements not measured).

Between sleep stages smaller periods of body-movements often occur which help the system recognize the change of stage.
Sleep pose: With a body mounted sensor device, the pose of the body-part where the sensor device is mounted can be calculated based on the direction of the measured gravitational vector. This enables the sensor device to recognize both whether the person is upright or laying down and whether he is laying on the side, stomach or back. This information is used as a discriminator when estimating sleep time.
Sleep time: recognizing the exact time of falling asleep can be difficult, and thus an estimation will be based on the amount of movements as well as the body pose. Thus, if the person is lying down and movements are far between, the person has likely fallen asleep.

Times for thresholds and movement frequencies are different from one person to the next, and are based also on parameters like age and BMI.

In one embodiment, data detected using the accelerometer is analyzed in epochs of a predefined duration (e.g. 5-seconds epochs). Hereafter, each epoch can be estimated to belong to a certain activity category. In one embodiment, the categories are characterized by intensity, wherein the activity intensity is calculated as the average vector magnitude of the high-pass filtered (e.g. three-dimensional) accelerometer measurements at a predefined sampling frequency (e.g. 5-20 Hz, such as 12 Hz) subtracting the noise present on each measurement axis. This is preferably done for each (e.g. 5 second) epoch. Hereby, it is possible to achieve a good indication of the intensity of the measurement. Accordingly, the detected intensity can be used to distinguish e.g. slow walking from fast walking.

In one embodiment, the category “resting” is estimated when the sensor is in a horizontal position (meaning that the leg is in a horizontal position) (+− approximately 45 degrees). Higher intensities indicate that the person wearing the sensor device is riding a bike, and signals are further analyzed to check this (see biking). The category “resting” can be seen as an indication of the patient being sedentary, sitting or lying down.

The category “standing” may be defined as a configuration, in which the sensor device is in a vertical position (meaning that the leg is in a vertical position—standing) (+− approximately 45 degrees) and the calculated intensity is lower than a predefined minimum movement threshold (e.g. 0.05-0.5 G such as 0.1 G). This category “standing” can be seen as an indication that the person wearing the sensor device is standing still with only very minor insignificant movements recognized.

The category “light activity” includes configurations, in which all movements in upright (vertical) position are associated with a vector magnitude above a predefined movement threshold, but below a predefined training intensity, wherein the movement is not recognized as falling into other categories. This will include epochs where an activity is starting towards the end of the epoch or walking just a few steps before again standing still. Also biking very slowly for shorter periods will fall into this category.

The category “biking” is estimated if a repeating pattern is recognized with a frequency above 0.2 Hz, where both legs move all the time and the measured movements are symmetrical. This is recognized based on pattern recognition. Also, the intensity has to be above a predefined measurement threshold. This category can be seen as an indication of the person wearing the sensor device doing a cyclic leg movement for more than 1-2 minutes (with pauses). Smaller biking intervals (trips) below 1 minute are not seen often, and will likely be detected as light activity.

The category “walking” is estimated if a repeating pattern with an intensity above the measurement threshold, but below the threshold for training activity, as well as a frequency above 0.2 Hz is recognized based on pattern recognition. As one leg at a time takes a step forward, and as the sensor is mounted on one leg, the measured movement has to be asymmetrical, and thus the pattern recognition does not yield biking. This category can be seen as an indication of the person wearing the sensor device walking for a predefined period (e.g. of 5-10 seconds) continuously. Shorter walking intervals (trips) below 3-9 seconds will likely be detected as light activity.

The category “training” is defined as a category used for any walking activity above the training intensity interval for a predefined period (e.g. of 3-9 seconds) continuously (which is not recognized as biking).

This category should be seen as an indication of the person wearing the sensor device running or doing high intensity training involving the leg. Shorter running intervals (trips) below 3-9 seconds will likely be detected as light activity or walking.

Steps taken: During light activity, walking or training, the number of steps is recognized based on an analysis in the frequency domain.

The method is a method for detecting if a sensor device is in contact with or in close proximity to the skin, wherein the method comprises the step of detecting temperature, acceleration and capacitance by using sensing elements, wherein the method comprises the step of detecting if the sensor device is in contact with or in close proximity to the skin of a user if at least two of the at least three measurements made by different sensing elements indicate that the sensor device is in contact with or in close proximity to the skin of a user.

Hereby, it is possible to provide a method that is more reliable than the prior art methods for detecting when a sensor is worn.

In one embodiment, the method comprises the step of detecting if the sensor device is in contact with or in close proximity to the skin of a user if at least three of the at least three measurements made by different sensing elements indicate that the sensor device is in contact with or in close proximity to the skin of a user. Hereby, it is possible to provide a very reliable method.

In one embodiment, the method comprises the step of saving measurement data and wirelessly transmitting the measurement data to a remote unit, wherein the sensor device has a unique identity (ID) and a predefined encryption key stored in the sensor device, wherein the method comprises the step of encrypting the measurement data using the encryption key before the measurement data is sent to the remote unit.

Hereby, it is possible to provide a method by which data can be safely transmitted using a non-secure communication protocol.

The antenna may be any suitable type of antenna. In an embodiment, the sensor device comprises a printed circuit board (PCB) having an integrated antenna. In one embodiment, the antenna may be a PCB trace antenna. Alternatively, the antenna may be a chip antenna.

The power source of the sensor device may be a battery. The battery may be a removable battery. In one embodiment, the battery is a rechargeable battery. In one embodiment, the power source may be an energy harvesting unit (e.g. piezo-mechanical harvesting, radiofrequency energy harvesting, thermoelectric harvesting (e.g. using a thermoelectric generator)). In one embodiment, the battery is a disposable battery that cannot be replaced with another battery because the sensor is arranged in a non-openable housing.

The one or more sensing elements are configured to provide measurement data. The sensing elements may include sensing elements for measuring any suitable parameter including temperature, capacitance, acceleration (or vibration) in one or more directions, heart rate, changes in skin redness and electrical conductivity.

In an embodiment, the detection unit comprises a capacitive proximity sensor. Hereby, the sensor device can provide useful information that indicates if the sensor device is in contact with or arranged in close proximity to the skin of a user.

In an embodiment the capacitive proximity sensor comprises a sensor plate electrically connected to a current sensor that is electrically connected to an oscillator connected to a direct current (DC) power supply. This type of capacitive proximity sensor is capable of detecting any object because any object has the ability to be electrically charged.

In one embodiment, the detection unit comprises an accelerometer. Hereby, the sensor device is capable of detecting if the user is active. Moreover, the sensor device may detect the posture of the user. Furthermore, the sensor device can detect if the sensor device is moved (e.g. when being detached from the user).

In one embodiment, the accelerometer is a single-axis accelerometer. In an embodiment, the accelerometer is a multi-axis accelerometer. In a particular embodiment, the accelerometer is a three-axis accelerometer.

In one embodiment, the detection unit comprises a temperature sensor. Hereby, the sensor device can provide temperature measurements that can be used to detect if the sensor is detached from the user or is in contact with the skin of the user.

In one embodiment the sensor device comprises a control unit configured to bring the sensor device into a power saving mode (sleep mode) when one or more of the measurement data exceeds or goes below a predefined threshold level. Hereby, it is possible to reduce the electricity consumption.

In one embodiment, the sensor device comprises a pocket structure configured to receive, contain and prevent the electronic components of the sensor device from coming into contact with water. Hereby, the sensor device can be applied by a user taking a shower.

In one embodiment, the sensor device is configured to detect the body posture of a user during sleep using data detected by the accelerometer. Hereby, the sensor device can be used to perform useful measurements of users during their sleep. Using data detected by the accelerometer it is possible to determine the orientation of the sensor and thus the posture of the user.

It may be an advantage that the sensor device is configured to identify the type of physical activity from a predefined list of activities using data detected by the accelerometer. Hereby, the sensor device can be used as an effective activity-monitoring tool. The data detected by the accelerometer can be analyzed in order to determine range of motion, motion frequency and other relevant parameters.

In an embodiment, the sensor device is provided with a disposable battery and the sensor is arranged in a non-openable housing. Hereby, it is meant that the housing is non-openable except through total destruction of one or more housing members. Thus, the disposable battery cannot be exchanged unless the housing of the sensor is destroyed.

In one embodiment, a method is suitable for transferring (wirelessly transmitting) measurement data obtained by a sensor device comprising one or more sensing elements configured to provide measurement data, wherein the method comprises the step of saving measurement data and wirelessly transmitting the measurement data to a remote unit, wherein the sensor device has a unique identity and a predefined encryption key stored in the sensor device, wherein the method comprises the step of encrypting the measurement data using the encryption key before the measurement data is sent to the remote unit.

Hereby, it is possible to provide a method for safely transmitting data using a non-secure communication protocol.

It may be an advantage that the sensor device comprises a memory (and that the method comprises the step of saving/storing the measurement data in encrypted form in the memory before sending the measurement data to the remote unit).

In one embodiment, the method comprises the step of detecting temperature and/or capacitance and/or acceleration and/or heart rate and/or changes in skin redness and/or electrical conductivity.

It may be an advantage that the method comprises the step of detecting if the sensor device is in contact with a user using a capacitive proximity sensor and/or an accelerometer and/or a temperature sensor.

In one embodiment, the method comprises the step of detecting if the sensor device is in contact with a user using a capacitive proximity sensor and an accelerometer and a temperature sensor, wherein it is concluded that the sensor device is in contact with a user when at least two of the three measurements indicate that the sensor device is in contact with a user.

In one embodiment, the sensor device is configured to be temporarily fixed in proximity of the body of a user (e.g. on the skin of the user). The sensor device may be configured to monitor health related parameters of physical activity of a user.

In one embodiment the sensor device comprises a PCB comprising at least one sensing element configured to provide measurement data relating to and/or representing physical activity.

The sensor device comprises a memory configured to store sensor measurements in an encrypted format. The sensor comprises a processor configured to encrypt detected measurement data. In one embodiment, the processor is adapted to transfer encrypted measurement data wirelessly. Throughout this document, the measurement data may also be referred to as sensor data, sensitive data or simply data.

In one embodiment, the sensor device comprises an electronic structure that is sealed inside a patch wherein the (thickness of the) patch is in the range 1-6 mm, such as 2-5 mm.

In an embodiment, the sensor device comprises a flexible printed circuit that can be adhered to the skin of the user with an adhesive patch material for more than about 24 hours.

In an embodiment, the sensor device is configured to wirelessly transfer encrypted data to a cloud system or similar system allowing only registered users to have access to it. This eliminates the risk of data being copied by unauthorized devices.

The sensor device is an affordable and disposable device that can be attached to the individual for more than about 24 hours without being noticeable. By having a flexible and sealed patch with a thickness up to 4 mm, it is possible for the individual to perform daily activities without any or with minimal discomfort.

In one embodiment, the sensor device comprises a link device configured to establish a link between one or more health personnel and the citizens. Hereby, it is possible to allowing trainers to provide feedback, and target the citizens if required.

The sensor device allows for big data management in a secure way, even under public connectivity, using encrypted data at all stages.

In one embodiment, a cloud system stores sensitive data from several users in a manner in which the data is encrypted, wherein only users having an access code are able to access the cloud and where users have restricted access according to their credentials.

Such a cloud system configuration allows the system to have two different types of users: user A/a first user meant to wear the device and generate the data and user B/a second user meant to access at least some of user A's collected data in order to analyze it, review it, manage it, etc. and provide direct feedback if necessary.

The sensor device is described with a set of features but can be complemented with the use of other sensors from other devices. The complementary data may be collected in the cloud system. Thus, a second/further device (also being worn by the user) can be used in order to have more precise information about the user, such as specific body posture.

In one embodiment, the method comprises the step of managing big data collected using a sensor device according to the invention. This allows analysis and identification of early stage diagnostics. The invention may be used as a tool for health professionals to efficiently follow up with larger patient groups and provide support. The sensor device is designed to register detailed activities like standing, walking, and exercises too slow for existing activity trackers to sense, but which nonetheless are important for health recovery.

In one embodiment, the method applies a sensor system comprising a sensor device according to the invention with a cloud system comprising a back-end, wherein the method comprises the following steps:
applying the sensor device to detect measurement data;
encrypting the measurement data using the encryption key stored in the sensor device;
sending the encrypted measurement data to the back-end;
applying the back-end to select the encryption key from a list of encryption keys on the basis of the unique identity of the sensor device;
applying the back-end to decrypt the encrypted measurement data from the sensor device using the selected encryption key.

### BRIEF DESCRIPTION OF THE DRAWINGS

The invention will become more fully understood from the detailed description given herein below. The accompanying drawings are given by way of illustration only, and thus, they are not limitative of the present invention. In the accompanying drawings:

FIG. 1A shows a schematic top view of a sensor device according to the invention;

FIG. 1B shows a schematic top view of another sensor device according to the invention;

FIG. 1C shows a schematic view of a sensor device according to the invention comprising a PCB sealed inside the sensor device;

FIG. 1D shows a diagram of connections between different components in the PCB shown in FIG. 1C;

FIG. 2A shows how a sensor device according to the invention transfers data to a cloud system through a remote unit;

FIG. 2B shows interaction between two users;

FIG. 3A shows the component layout of a cloud system;

FIG. 3B shows an example of how information provided by sensor devices according to the invention can be provided;

FIG. 4A shows a flowchart that illustrates how to detect if the sensor device is attached to a user;

FIG. 4B shows an exemplary design of a sensor;

FIG. 5 shows a flowchart illustrating how data is encrypted and stored on a memory of a sensor device;

FIG. 6A shows a graph illustrating the capacitance detected by a sensor device as a function of time;

FIG. 6B shows a graph illustrating the temperature detected by a sensor device as a function of time;

FIG. 6C shows a graph illustrating the activity level detected by a sensor device as a function of time;

FIG. 7A shows how data is transferred from a sensor device to a cloud system;

FIG. 7B shows a sensor device within a mounting structure, comprising a pocket structure, that prevents exposure of the sensor to water;

FIG. 8A shows a sensor device according to the invention attached to the arm of a user; and

FIG. 8B shows a close-up view of the sensor device shown in FIG. 8A.

### DETAILED DESCRIPTION

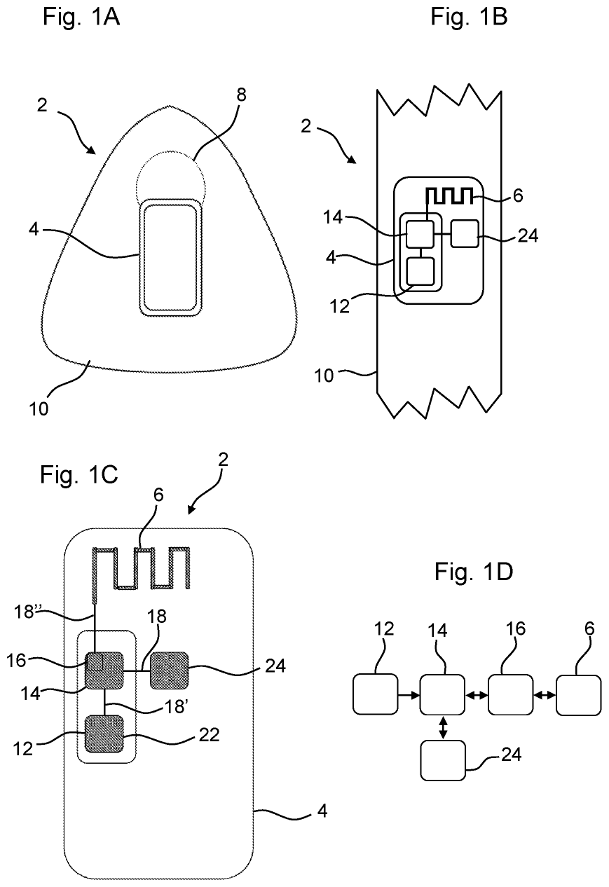

Referring now in detail to the drawings for the purpose of illustrating embodiments of the present invention, a sensor device 2 of the present invention is illustrated in FIG. 1A.

FIG. 1A illustrates a schematic top view of a sensor device 2 according to the invention. The sensor device 2 comprises a PCB 4 and a break detector 8 formed as a wire. The sensor device 2 moreover comprises a mounting structure 10 configured to be detachably attached to the skin of a user. The mounting structure 10 seals the PCB 4 and hereby protects the PCB against water. Therefore, the sensor device 2 can be worn when taking a shower without affecting the ability to perform the sensor measurements that the sensor device 2 is configured to carry out.

The break detector 8 is integrated in the mounting structure 10 and thus arranged in such a manner that it will break due to the deformation of the mounting structure 10 if the sensor device 2 is removed from the user.

The sensor device is configured to detect data indicative of activity, lifestyle, and certain predetermined parameters of the individual (user) that wears the sensor device 2. The sensor device 2 is configured to collect and wirelessly transmit encrypted data. In a preferred embodiment, the sensor device 2 is flexible and disposable. It may be an advantage if the thickness of the sensor device 2 has a thickness that does not exceed 4 mm.

The sensor device 2 may comprise a patch configured to communicate (two-way communication) wirelessly with a storage facility such as a database either directly or through the use of a remote monitoring unit, such as a smartphone or tablet via Bluetooth Classic, Bluetooth Low Energy, or another suitable wireless communications protocol.

The data transmitted by the sensor device 2 is encrypted according to one or more industry standard encryption formats. The stored data may preferably be analyzed and presented to a user through different formats that may differ according to the user's credentials and purpose (e.g. to give feedback in order to improve a user's health).

The sensor device 2 may be attached to the skin of a user in different positions. The sensor device 2 may be configured to be attached to the skin of a user at a position either 5 to 20 cm above the knee on the inner, front, or outer side of the thigh, on the foot, or on the inner, front or outer side of the lower part of the leg. Other possible locations could e.g. be on the upper body e.g. the upper stomach, the chest, or the back. The sensor device 2 may be adhered with a pressure-sensitive adhesive (PSA) designed to form a temporary bond, so it can be worn for a longer period of time e.g. for more than about 24 hours and be removed when desired. The sensor device 2 may be a single use and disposable device. The PSA is an adhesive that does not damage the skin when the sensor device 2 needs to be removed.

The sensor device 2 is configured to determine if the sensor device 2 is attached to or detached from the body of the user. The sensor device 2 comprises a mechanical break detector 8 configured to provide information in case the sensor device 2 is detached from a user.

The sensor device 2 may comprise a mechanical break detector 8 formed as a metal wire or connector that closes a circuit having an end structure that is attached to an input port of the processor (or processing unit or integrated circuit, IC) of the sensor device 2, wherein the opposite end structure is attached to an IC power-supply pin: either ground or the positive supply voltage pin (V_{CC}) of the IC, respectively. If the patch is detached, the wire 3 will break, which enables device 2 to measure directly that the connection is broken and store the event that the patch has been detached.

FIG. 1B illustrates a schematic top view of another sensor device 2 according to the invention. The sensor device 2 comprises a mounting structure 10 formed as a band configured to be worn around the ankle or wrist. Only a portion of the band is shown in FIG. 1B. Thus, the band may be longer hereby enabling the user to wear it around the ankle or wrist.

The sensor device 2 comprises a PCB 4 comprising an antenna 6 integrated therein as a PCB trace antenna 6. The PCB comprises a processor 14 electrically connected to the antenna 6, a memory 24 and a sensor element 12. The sensor device 2 may comprise a plurality of sensor elements 12 even though only a single sensing element 12 is shown. The processor 14 is configured to process data (including encrypting data) received from the sensor element 12. At the same time, the processor 14 functions as a communication unit (shown in FIG. 1C) that is configured to wirelessly transmit signals to a remote receiver.

FIG. 1C illustrates a schematic view of a sensor device 2 according to the invention comprising a PCB sealed inside the sensor device 2. The sensor device 2 comprises a mounting structure (not shown). The sensor device moreover comprises a PCB 4 provided with an antenna 6 integrated therein as a PCB trace antenna 6. The PCB further comprises a processor 14 electrically connected to the antenna 6 by means of a connector 18″ and a memory 24 connected to the processor 14 by means of a connector 18. The processor 14 comprises a communication unit 16 configured to wirelessly transmit encrypted data to a remote unit. The PCB comprises a sensor element 12 electrically connected to the processor 14 by means of a connector 18′.

The sensor device 2 may comprise a plurality of sensor elements 12 even though only a single sensing element 12 is shown. The processor 14 is configured to process data (including encrypting data) received from the sensor element 12. The processor 14 functions as a communication unit (shown in FIG. 1C) that is configured to wirelessly transmit signals to a remote receiver. The processor 14 is configured to encrypt the measurement data before the measurement data is transferred to the remote unit.

The PCB containing the sensing element 12 may be sealed inside the sensor device 2. Direct contact with the skin of a user may be an advantage. Accordingly, the sensor device 2 may preferably comprise a mounting structure comprising an adhesive portion adapted to be attached to the skin of a user. In a preferred embodiment, the processor 14 is electrically connected to at least two different sensing elements 12 (even though only one is shown in FIG. 1C).

In one embodiment, the processor 14 is electrically connected to a temperature sensor and a multi-axis accelerometer. It is preferred that the sensor device 2 is flexible. This may be achieved by producing the PCB in a flexible printing material like a polyimide film (e.g. “Kapton” or “FR-4”). It may be an advantage that the thickness of the PCB is 0.4 mm or less.

The PCB 4 may be sealed using a silicone material, a medical grade silicone with shore value of A30-A80, or a plastic material. The PCB 4 may be located on the top of a layer of medical tape with PSA that hereby will give the final form of the sensor device 2. The purpose of the medical tape arranged below the PCB 4 of the sensor device 2 is to fix the sensor device 2 firmly to the skin of a user and to transport moisture away from below the sensor device 2.

In one embodiment, the sensor device 2 comprises a top patch layer configured to protect the skin and sensor device 2 against moisture as well as fixing the sensor device 2 more firmly.

The sensor device 2 is configured to generate measurement data that indicates the type of activity by analyzing the type of movement performed by the user. The processor 14 is configured to analyze the sensor signal detected by an accelerometer 12 and identifies the type of activity. The processor 14 is configured to encrypt and store the encrypted measurement data in the memory 24. Accordingly, later the encrypted measurement data can be transmitted wirelessly to a remote unit through the antenna 6.

FIG. 1D illustrates a diagram of connections between different components (the antenna 6, the sensing element 12, the processor 14, the communication unit 16 and the memory 24) of the PCB shown in FIG. 1C. The processor 14 receives measurement data from the sensor element 12. The processor 14 encrypts the received measurement data that is stored in encrypted form in the memory (24). Encrypted data is also received by the processor from the memory 24. The processor 16 is connected to the communication unit 16 that is capable of wirelessly transmitting encrypted data to a remote unit and optionally to receive wireless information sent by an external device via the antenna 6.

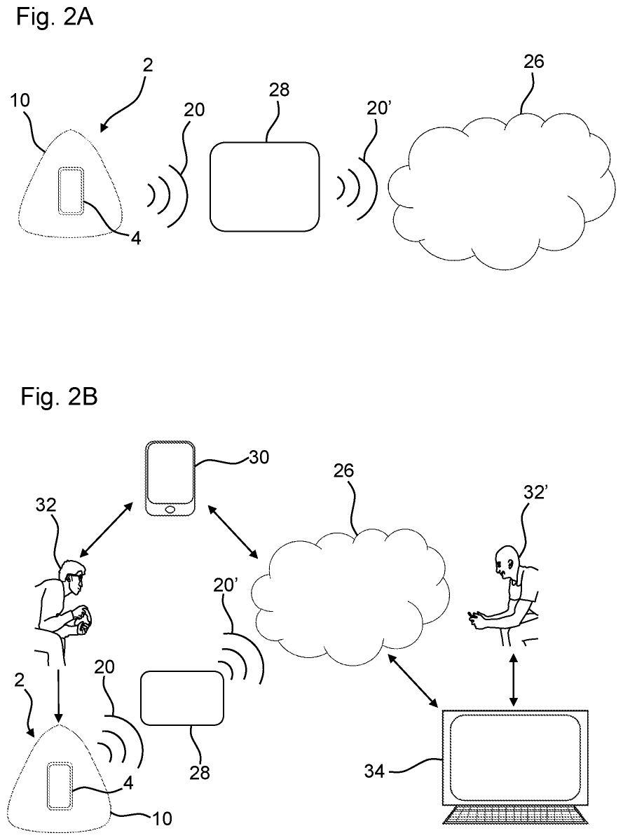

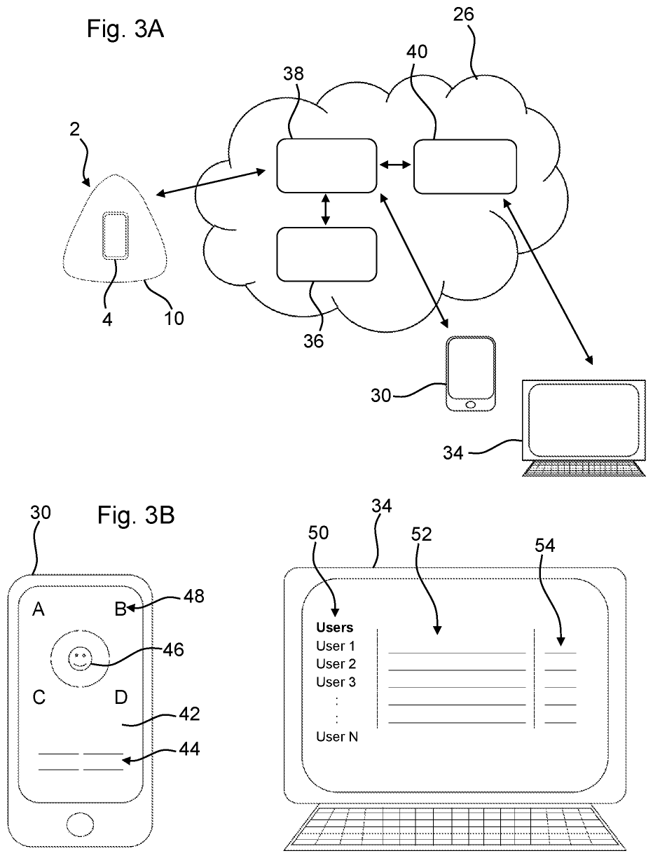

In an embodiment, the sensor device 2 comprises a sealed PCB arranged in the sensor device 2. The PCB contains at least one sensing element 12 that is connected to processor 14. When a digital signal is generated in the sensing element 12, that signal is sent to the processor 14, which will process and identify the type of data. The processor 14 encrypts the data and sends the encrypted data to the memory (e.g. a flash type memory) 24. All sensing elements 12 are configured to generate digital signals and are connected to the processor 14. Both the processor 14 and the memory 24 can have input and/or output circuitry, which allows for the transfer of digital data between the processor 14 and the memory 24. Thus, memory 24 will collect encrypted data representing the user's activity data over a specific predetermined amount of time. This data can be sent to a database such as a cloud system (see FIG. 3A), through a remote monitoring unit, e.g. as shown in FIG. 2A, where the data will be stored, and be accessible for a registered user.

In order to make the transfer possible, the processor 14 can manage and process the collected data in the memory 24 and can furthermore send it wirelessly via a communication unit 16 (comprising a transceiver) and an antenna 6 to the remote monitoring unit. Any suitable communication protocol such as Bluetooth can be applied to carry out the communication.

The PCB of the sensor device 2 may comprise input and/or output circuitry adapted to output and receive as input certain data signals that can be received by a transceiver connected between the antenna 6 and the processor 14. Additionally, when the collected (and encrypted) data is sent to the cloud system 26 (see FIG. 2A, FIG. 2B or FIG. 3A) through an interaction with a remote monitoring unit, device 2 can be as far away as the wireless connection enables. The transmitter and receiver may be embodied by any suitable transceiver.

FIG. 2A illustrates how a sensor device 2 (having a PCB 4) according to the invention transfers data in the form of wireless signals 20 to a cloud system 26 through a remote unit 28. The remote unit 28 may be a smartphone, a tablet or another device capable of receiving the wireless signals 20. The remote unit 28 sends wireless signals 20′ to the cloud system 26. It should be noted that the communication between the remote unit 28 and the cloud system 26 may be carried out using a wired connection.

FIG. 2B illustrates an interaction between a first user 32 and a second user 32′. In one embodiment, support is provided for two different types of users. FIG. 2B shows an overview of how the two groups differ in their use of the sensor device 2. The first user type 32 is meant to wear the sensor device 2 and generate data. This user 32, will set up a profile through the use of a remote unit 28, such as a smartphone or tablet, that holds an external device (e.g. a personal digital assistant remote) 30, such as an app, providing e.g. information consisting of name; age; weight; height; and mobility level, parameter assessed using a mobility scale from well-functioning to support needed.

User type 32 will e.g. also set up the account with a personal password and will be able to access her/his own generated data as a viewer. A second type of user, user 32′, is possible. User 32′, won't generate data through the sensor device 2 but can have access to the personal data through a display unit 34, such as a personal computer, that has access to a relevant electronic network, such as the internet, and will set the personal goals that user 32 should achieve.

In one embodiment, user 32′ is allowed to be in charge of the health progress of user 32. Thus, user 32′ may be in charge of more than one user 32. This implies that user 32′ may have access to the collected data by the sensor device 2 from more than one user 32. By having access to more than one user's collected data, and comparing the results, user 32′ can identify common patterns in the activities and behaviors of the monitored users and actively give personalized feedback. This allows for the management of collected data in a safe environment and user 32′ can manage big amounts of data using one platform. User 32′ can e.g. be part of a health care organization, an insurance company, or other organization in need of monitoring the health progress of more than one individual.

In one embodiment, both user 32 and 32′ have access to see the collected data of user 32 in different visualization modes such as graphs, a table, or other visualization types. The comparison and management of different user's collected data allows for big data management.

FIG. 3A illustrates the component layout of a cloud system 26. The cloud system 26 comprises a back-end 38, a front-end 40, and a database 36. Overall, the cloud system 26 can store sensitive data collected by the use of a sensor device 2 in an encrypted manner. When the data is first transferred from the sensor device 2 through a remote monitoring unit (see FIG. 2B), the data is encrypted to secure safety of sensitive data passed through an insecure connection. When a user wants to access the data, the user will e.g. be provided with a specific code that will give the user access to the data according to the user's credentials.

In one embodiment, a platform is provided to create a database. Once the data is transferred to the cloud system 26, the encrypted data is de-encrypted by the back-end 38 as described previously and stored in the encrypted database 36. The collected data stored in the database 36 may consist of metadata and sensor measurements.

The monitored sensor data may be used to estimate relevant physical activity parameters for each user.

In one embodiment, the cloud system 26 works in the following manner. When the sensitive data from the user is collected with the use of the sensor device 2 and transferred through the use of a remote monitoring unit, the data is transferred to the back-end 38 of the cloud system 26. When received at the back-end 38, the data is decrypted and transferred to a secure encrypted database 36. For processing purposes, the data needs to be transferred to a frontend 40, in order to display and manage the data using a display unit 34, such as a personal computer, that has access to a relevant electronic network, such as the Internet. As described, the sensitive data is always encrypted to ensure safety, so both back-end 38 and front-end 40 will contain the data encrypted and only users with an access code will be able to access the system.

The front-end 40 can present the collected data and receive detailed input through the use of a display unit 34 that has access to a relevant electronic network such as the internet. The back-end 38 is responsible for providing an application interface or similar software for accessing and storing data into the underlying data storage database 36.

A user can also access the data collected through the use of a remote monitoring unit (see FIG. 2B) such as a smartphone or tablet using a relevant electronic network such as the internet. The external device (remote monitoring unit) 30 will communicate directly with the back-end 38. If the user has access to the collected data through the front-end 40, thus by the use of a relevant electronic network such as the Internet, the user can manage the collected sensitive data that the user has access to depending on the user's credentials.

Referring to the sensitive data collected in the cloud system 26, all information is encrypted to protect the user's privacy. In one embodiment, when a user wants to access the collected data, the user has a secure two-factor login to the system, which will enable the user to view the data in the secure front-end 40. Thus, allowing the decryption of specific data for viewing.

In one embodiment, the sensor device 2 is configured to ensure that transmission to the remote monitoring unit of the collected data is activated when the sensor device 2 interacts wirelessly with a personal digital assistant remote (see FIG. 3A and FIG. 3B). This may be achieved by using an app called “SENS motion”, that is active in the background of the remote monitoring unit 28, such as a smartphone or tablet with access to the relevant electronic network, such as the Internet. This process does not require wires or pairing between the sensor device 2 and the personal digital assistant remote 30.

As the sensitive data collected by means of the sensor device 2 is encrypted as it is saved to the memory (internal storage) 24, the transmitted data will still be encrypted and thus safe from external and/or non-registered or unauthorized persons even if the connection as shown in FIG. 2A is public or not safe.

The method of data encryption could be, but is not limited to, a public private cryptographic key encryption scheme. This allows for the private key to only be known by the cloud system 26 while the public key is transferred over the wireless connection and used for the encryption of data on the sensor device 2 (therefore it is significant that the sensor device 2 comprises a receiver (part of the communication unit 16 shown in FIG. 1C, FIG. 1D).

The remote monitoring unit 28 that may comprise a personal digital assistant remote may be configured to automatically give access to the relevant electronic network for any reachable device via Bluetooth or the like. Once the sensor device 2 has access to the relevant electronic network, the data is transferred and stored on a cloud system 26, which will transmit back to the sensor device 2 a command or request to delete the transmitted data from the memory 24.

In one embodiment, the method of transferring data, which combines different features such as, the wearable detection by sensing elements 12, encryption of the data, and the use of a personal digital assistant remote 30, such as the app called “SENS motion”, leads to a transfer without need of pairing or secure connection.

In one embodiment, the method makes it possible to identify a user that is sending data. This identification may be based on a unique ID of the sensor device 2, and thus, facilitates the process of storing the data into the cloud system 26. As mentioned, the data transferred from the sensor device 2 may be encrypted when it is transferred and only decrypted after transfer as it is stored into the secure database 36 within the cloud system 26. Following this procedure, the transfer method is protected from a possible external injection of fake data or extraction of sensitive data.

FIG. 3B illustrates an example of how information provided by sensor devices according to the invention can be provided. A first user applies an external device (a personal digital assistant remote) 30 to receive information. The external device 30 comprises a display 42. The first user may have first a set of goals set by another user, accessible through a menu 44. The first user can also see the different activities A, B, C, D that are being monitored and the progress accomplished in real time compared to the daily goals set by the second user. The number of activities A, B, C, D displayed may vary according to the number of activities that the second user wants to monitor for the first user.

The first user can receive motivational feedback 46 depending on the daily performance achieved or progress towards the set goals. The typically monitored and displayed data include, but not limited to, the time spent, number of repetitions and motion intensity in a number of activity categories such as walking, standing, cycling, or doing a number of different exercises like sitting down standing up or static strength exercises like leg press, etc. Data is both available as an overview of the performance over a defined period of time and at a specific point in time on a second to second level.

FIG. 3B illustrates an example of the display of information shown through a personal digital assistant remote 30 such as an app running on a smartphone or a tablet, as well as the visualization through a display unit 34, such as a computer, where the comparison takes place. The information will differ from a first user 32 to a second user 32′ (shown in FIG. 2B), as user 32 has restricted access being shown only the data generated by user 32. User 32 is meant to log in to the system through a back-end 38 with the personal digital assistant remote 30 and user 32′ is meant to log in to the system through a front-end 40 with a display unit 34.

As seen in FIG. 3B, the display for user 32′ through a display unit 34 can allow the comparison and management of sensitive data for more than one user 32. Thus, user 32′, can compare easily the performance of the various (first type of) users between each other as well as with each user's individual goals. The comparison may e.g. contain the different user 32's name 50 with each daily performance 52, during every relevant day of the week, and a summary 54 section that user 32′ can use to detect the user 32 most in need of active feedback.

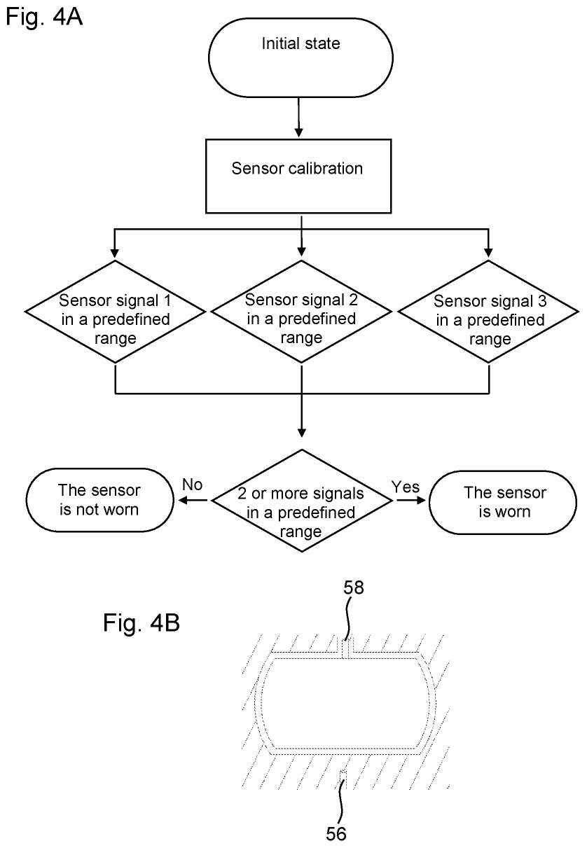

FIG. 4A illustrates a flowchart that illustrates how to detect if the sensor device is attached to a user. It can be seen that a sensor calibration is carried out as the first step after the initial state. Hereafter, each of the sensing elements determines if the measurement data (sensor signal 1, sensor signal 2 and sensor signal 3) is in a predefined range. This predefined range is selected in such a manner that the sensor device is assumed to be attached to the skin of a user.

If two or more of the sensor signals are within a predefined range, it is concluded that the sensor is attached to the skin of a user. On the other hand, if less than two of the sensor signals are within a predefined range, it is concluded that the sensor is not attached to the skin of a user.

In one embodiment, sensor signal 1 is measured by an accelerometer (e.g. a multi-axis accelerometer). If no accelerations are measured for a longer time period, it indicates that the sensor device is detached from the user. In a preferred embodiment, sensor signal 2 is measured by a capacitive proximity sensor that is capable of detecting if the sensor device is in contact with or arranged in close proximity to the skin of a user. In one embodiment, sensor signal 3 is measured by a temperature sensor. If the temperature is no longer within a predefined temperature interval (e.g. 25-30° C., e.g. approximately 27° C.) or if the temperature suddenly decreases, it indicates that the sensor device is removed from the user. FIG. 4B illustrates design of a sensor device. The sensor device comprises a ground terminal 56 and a common collector voltage (V_{CC}) terminal.

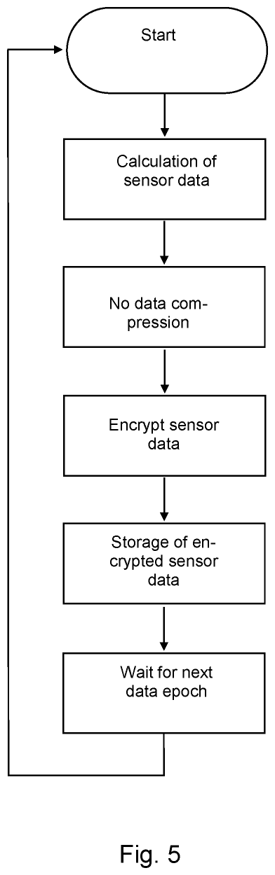

FIG. 5 illustrates a flowchart illustrating how data is encrypted and stored on a memory of a sensor device. The sensing elements provide digital inputs (data) to the processor. These data are encrypted by the processor and hereafter stored in the memory. Hereafter the processor waits for the next data epoch from the sensing elements. The flowchart shows a method for handling accelerometer data. When the accelerometer sensor has stored a number of movements, it interrupts the processor waking this component from its sleep to prepare for the transfer of sensor data. Data is directly received by the processor and will hereafter be processed and encrypted be stored in the memory (e.g. Flash).

Accordingly, this aspect defines a method which, once the sensor is mounted, enables data to only be stored based on the actual dynamic range of the accelerations. If the movements are powerful, the full dynamic range of the data signal is required to digitally represent the motion measured. If, however, movements are slower and thus measured acceleration dynamics lower, a significant data compression/reduction is achieved by only storing the relevant dynamic range of the signal—the signal difference from one measurement to the next. E.g. if the full range is −8G to 8G represented by 10 bit per accelerometer axis (3 axis) and the actual signal dynamics during a relevant epoch time frame (e.g. 5 seconds) is only within a −1G to 1G window, each signal (axis) can be represented by only 7 bits instead of 10, meaning a 30% reduction of the data stored. Fewer data to store also entails fewer data to transmit saving power consumption and bandwidth. If no, or little, dynamic accelerations (e.g. below a set acceleration threshold window) are identified by the accelerometer then the processor will completely stop the process of encrypting and storing the data in the storage thereby entering a power saving sleep-mode. The accelerometer will ‘wake up’ the processor again when future movement is detected by the accelerometer outside the set threshold window.

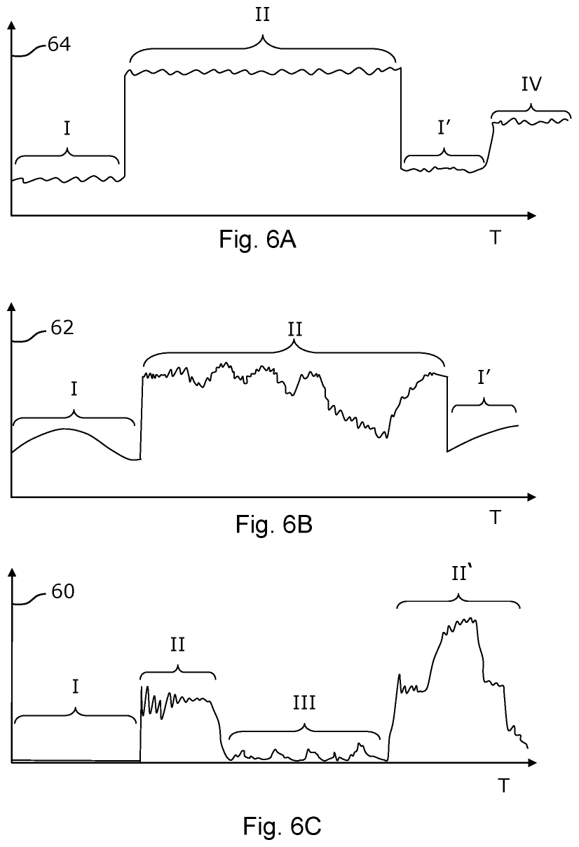

FIG. 6A illustrates a graph illustrating the capacitance detected by a sensor device as function of time. During a first time-period (dismounted sensor mode) I a low capacitance is detected, indicating that the sensor is not in contact with the skin of a user or in close proximity to the user. During a second time-period (mounted sensor mode) II a higher capacitance is detected, indicating that the sensor is now in contact with the skin of a user or in close proximity to the user. During a third time-period (dismounted sensor mode) I′ a low capacitance is detected, indicating that the sensor is not in contact with the skin of a user or in close proximity to the user. During a fourth time-period IV a slightly higher capacitance is detected, indicating that the sensor is moved to another surface.

FIG. 6B illustrates a graph illustrating the temperature 62 detected by a sensor device as a function of time T. During a first time-period (dismounted sensor mode) I a varying low temperature is detected, indicating that the sensor is not in contact with the skin of a user or in close proximity to the user. During a second time-period (mounted sensor mode) II a higher rather constant temperature is detected, indicating that the sensor is now in contact with the skin of a user or in close proximity to the user. During a third time-period (dismounted sensor mode) I′ an increasing low temperature is detected, indicating that the sensor is not in contact with the skin of a user or in close proximity to the user.

FIG. 6C illustrates a graph illustrating the activity level 60 detected by a sensor device as function of time T. During a first time-period (dismounted sensor mode) I a close-to-zero activity level is detected, indicating that the sensor is not in contact with the skin of a user or in close proximity to the user. During a second time-period (mounted sensor mode) II a higher varying activity level is detected, indicating that the sensor is now in contact with the skin of a user. During a third time-period (sleeping mode) III a lower varying activity level is detected, indicating that the user wearing the sensor device is sleeping or is basically not moving. During a fourth time-period (mounted sensor mode) II′ a higher varying activity level is detected, indicating that the sensor is now in contact with the skin of a user.

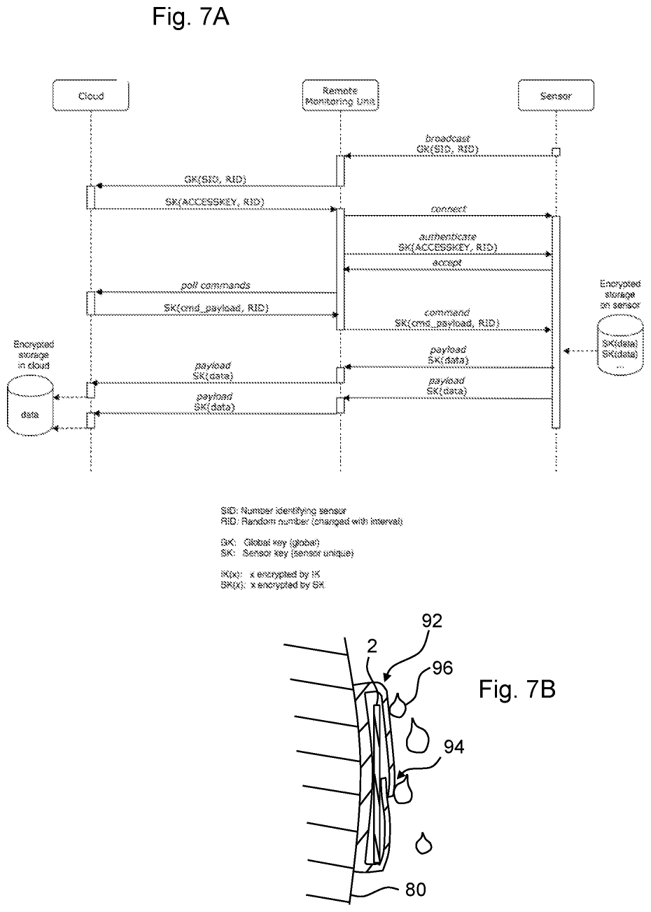

FIG. 7A illustrates how data is transferred from a sensor device to a cloud system. The method of securely identifying a sensor and securely transferring data from the sensor is as follows. The cloud system and sensors have a shared global encryption key GK, required to be able to identify a sensor. Each sensor has a static identity number SID, a random identity number RID that changes periodically e.g. every 30 minutes, a unique encryption key SK, and a unique key ACCESSKEY. The cloud system knows the corresponding SK and ACCESSKEY of all sensors based on their SID. A sensor will wirelessly broadcast its combined identity (SID, RID) encrypted with GK allowing a remote monitoring unit to discover a sensor but the remote monitoring unit will be unable to establish the sensor's identity and track it. GK (SID, RID) is sent to the cloud system which has the GK and can extract (SID, RID). It will provide an authentication payload consisting of (ACCESSKEY, RID) encrypted with SK that must be sent to the sensor to be allowed to connect. The sensor will decrypt this payload and verify the correct (ACCESSKEY, RID). This payload is valid only until the sensor invalidates a RID. All data between sensor and cloud system is encrypted with the SK thus the remote monitoring unit is unable to intercept it.

FIG. 7B illustrates a sensor device 2 comprising a mounting structure comprising a pocket structure 92 configured to receive, contain and prevent the electronic components of the sensor device 2 from coming into contact with water 96. Accordingly, the sensor device 2 can be applied by a user taking a shower. The pocket structure 92 comprises an overlapping portion 94.

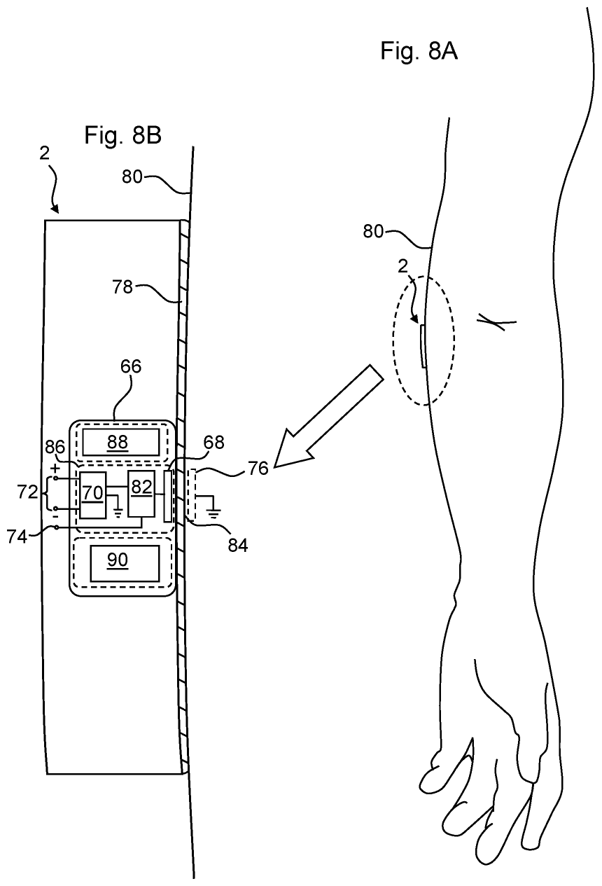

FIG. 8A illustrates a sensor device 2 according to the invention attached to the arm of a user. The sensor device 2 is attached to the skin 80 of a user.

FIG. 8B illustrates a close-up view of the sensor device 2 shown in FIG. 8A. the sensor device 2 comprises a contact detection unit 66 comprising three sensing elements: a capacitive proximity sensor 86, an accelerometer 88 and a temperature sensor 90. These sensors 86, 88, 90 are provided on a PCB 4 of the sensor device 2. The capacitive-proximity sensor 86 can provide information enabling assessment of whether the sensor device 2 is mounted on the skin 80 of a user or not by detecting a measured change in the capacitance. The capacitance proximity sensor 86 comprises a sensor plate 68 arranged close to the end surface of the sensor device 2. A current sensor 82 is electrically connected to the sensor plate. The current sensor 82 is connected to an output terminal 74 and to an oscillator 70 that is electrically connected to a power supply 72.

The sensor device 2 is attached to the skin 80 by means of an adhesive portion 78. Hereby a dielectric portion 84 is provided between the sensor plate 68 and a user (a theoretic capacitor plate indicated by a dotted rectangle).

The capacitive proximity sensor 86 detects that the capacitance of the sensor plate 68 increases when near to the skin 80 of the user or other surfaces containing water or metal. The capacitance measured will stay fairly constant with smaller variations within a certain dynamic range while the sensor is mounted. If the capacitance decreases, the sensor device 2 could potentially be in the process of being dismounted. This would trigger a further analysis on whether the sensor device 2 is really being dismounted or not. The same would result from a certain increase in measured capacitance resulting from the sensor device 2 being mounted.

To detect whether the sensor device 2 is mounted or not, these dynamics can be modeled e.g. using neural network machine learning using a large number of sample data for sensors known to be worn or not. The model could e.g. be based on the representation of sensor data as a histogram of the distribution of sensor values, constructed based on a sliding window approach. Bayesian models or simple thresholds could also be used in the same way based on mathematical models of the sensor dynamics (e.g. linear or non-linear distribution of sensor values in the histogram as well as the mean value for a certain number of measurements.

The temperature sensor 90 measures the surface temperature a few millimeters above the skin 80, at the area in which the sensor device 2 is mounted. By modeling the dynamics of the temperature signal, the processor can detect irregularities resulting from the sensor device 2 being dismounted. When mounted, the temperature is usually a bit above about 27° C. when the user is indoors. The temperature may vary a lot if the user also stays outside, works out, or e.g. sleeps with one leg inside or outside the blanket at night. If the sensor device 2 is not mounted, the temperature tends to be rather constant, with only smaller and fairly slow variations in temperature.

When the sensor device 2 is being mounted or dismounted, a fast temperature change will be accepted. By checking for fast temperature variations, whether the temperature is fluctuating as would normally be the case when mounted or fairly constant as dismounted will give an indication on whether the sensor device 2 is mounted or not. To detect whether the sensor device 2 is mounted or not, these dynamics can e.g. be modeled using neural network machine learning using a large number of sample data for sensors known to be worn or not.

The model could e.g. be based on the representation of measurement data (sensor data) as a histogram of the distribution of sensor values constructed or based on a sliding window approach. Bayesian models or simple thresholds could also be used in the same way based on mathematical models of the sensor dynamics (e.g. linear or non-linear distribution of sensor values in the histogram as well as the mean value for a certain number of measurements).

The dynamics of a normal person's wear cycle for accelerometer measurements consist of a minimum of activity, even at night there is still activity as the person turns in bed during a normal sleep cycle and even due to the small accelerations due to the blood circulating in the veins. These dynamics may be used as a parameter when measuring whether the sensor device is mounted or has been dismounted.

If the sensor device 2 is subject to no, few, or smaller accelerations or the sensor device 2 is being flipped or rotated in the gravity field, this is likely a result of the sensor being dismounted. On the other hand, if the sensor device is subject to several (repeating) smaller accelerations, the sensor device 2 is likely mounted. The frequency response can furthermore be calculated to check whether the movements resemble human motion like standing (smaller movements), walking (motion between about 0.3 and about 1.8 Hz), running (motion between about 1.8 Hz and about 5 Hz), cycling (between about 0.5 and about 5 Hz).

Examples of the dynamics of the sensors are shown in FIG. 6A, FIG. 6B and FIG. 6C and may in some embodiments be modeled using statistical Bayesian models, neural networks machine learning or simple signal thresholds. Neural networks can be trained using sensor signals from known data sources, e.g. data from an accelerometer known to be worn. Bayesian models will require a good mathematical representation of the sensor signal when worn or not. Simple thresholds governing whether the signal or signal dynamics is higher or lower than a set value are easy to implement, but will require calibration of sensor values, while having a hard time detecting more complex signal characteristics.

It is possible to combine data from two or more of these sources to calculate whether the sensor is mounted or not. This is in some embodiments done through the algorithm described with reference to FIG. 2A. The dynamics of each signal are analyzed over a period of time with given intervals on the cloud based backend. E.g. if the accelerations recorded suggest that the sensor device 2 has been dismounted, the signal from the temperature and/or capacitive sensor is analyzed to check whether the dynamics of these sensors also resemble a dismounted or mounted sensor device 2.

This feature is enabled since the processor is the link between the sensing element(s) and the memory. Accordingly, the processor can enter into a special sleep mode and will only wake-up again when interrupted by the accelerometer (when activity is detected). When the accelerometer is exposed to accelerations above a certain threshold window, an interrupt is sent to the processor, waking it up to process the data from the accelerometer.

Additionally, the processor is at least in some embodiments configured to identify different activities from the collected data down to a second to second level, e.g. using a sliding window algorithm approach. Such activities include sleep patterns, walking, running, cycling, sitting, lying down among others. Thus, if sensor device 2 is combined with a second sensor device, other postural activities can be identified, such as sitting, and body posture. The posture of the body part where the sensor device 2 is mounted can be calculated based on accelerometer data, as the position of the sensor device 2 on the body of a first user is known and the gravitational vector pulling 1G of constant accelerations towards the ground. This allows for the calculation of the body posture during sleep based on the posture of the data of the sensor device 2.

Allowing a second to second indication of the posture such as but not limited to laying on back, left side, right side or stomach. This data allows for detailed analysis of sleeping patterns by registering a vector magnitude of movements in different body postures.

In some embodiments, sensor device 2 is used in combination with other sensors meant to monitor other parameters than those disclosed. For example, for monitoring the effect of physical activity during a specific amount of time while monitoring other parameters like blood pressure that does not require constant monitoring.

### LIST OF REFERENCE NUMERALS

2 Sensor
4 Printed circuit board
6 Antenna
8 Break detector
10 Mounting structure
12 Sensing element
14 Processor
16 Communication unit (transceiver/transmitter)
18, 18′, 18″ Connector
20, 20′ Wireless signal
22 Electronic component
24 Memory
26 Cloud system
28 Remote unit
30 External device
32, 32′ User
34 Display unit
36 Database
38 Back-end
40 Front-end
42 Display
44 Menu
46 Feedback
48 Activity
50 Name
52 Performance
54 Summary
56 Ground
58 Common Collector Voltage (V_{CC})
60 Activity level
62 Temperature
64 Capacitance (e.g. measured in Pico Farad)
66 Contact detection unit (e.g. a capacitive proximity sensor)
68 Sensor plate
70 Oscillator
72 Power supply
74 Output (DC)
76 Theoretic capacitor plate
78 Adhesive portion
80 Skin
82 Current sensor
84 Dielectric portion
86 Capacitive proximity sensor
88 Accelerometer
90 Temperature sensor
92 Pocket structure
94 Overlapping portion
96 Water
T Time
I, I′ Dismounted sensor mode
II, II′ Mounted sensor mode
III Sleeping mode
IV Sensor moved to another surface

What is claimed is:

1. A sensor device comprising:
a processor;
a memory;
an antenna;
a power source;
at least three sensing elements configured to provide at least three measurements made by different sensing elements and to store the measurement data in the memory;
a communication unit configured to transfer data from the at least three sensing elements and data stored in the memory to a remote unit; and
a contact detection unit configured to detect that the sensor device is in contact with or in close proximity to the skin of a user when at least two of the at least three measurements made by different sensing elements indicate that the sensor device is in contact with or in close proximity to the skin of a user.

2. The sensor device of claim 1, wherein the contact detection unit comprises a capacitive proximity sensor, an accelerometer and a temperature sensor.

3. The sensor device of claim 2, wherein the sensor device is configured to detect a body posture of a user during sleep using data detected by the accelerometer.

4. The sensor device of claim 1, wherein the sensor device is configured to determine that the sensor device is in contact with or in close proximity to the skin of a user if three of the at least three measurements indicate that the sensor device is in contact with or in close proximity to the skin of a user.

5. The sensor device of claim 1, wherein the sensor device has a unique identity and a predefined encryption key stored in the sensor device, wherein the sensor device is configured to encrypt the measurement data using the encryption key before the measurement data is transferred to the remote unit.

6. The sensor device of claim 1, wherein the at least three sensing elements are configured to measure heart rate and/or changes in skin redness and/or electrical conductivity.

7. The sensor device of claim 1, wherein the sensor device comprises a mounting structure to mount the sensor device in contact with or in close proximity to the skin of a user.

8. The sensor device of claim 7, wherein the mounting structure comprises an adhesive portion configured to be detachably attached to the skin of a user.

9. The sensor device of claim 1, wherein the sensor device comprises a break detector arranged and configured to break if the sensor device is detached from a user.

10. The sensor device of claim 1, wherein the sensor device is contained within a pocket structure.

11. The sensor device of claim 1, wherein the sensor device is disposable and integrated in a patch.

12. A method for detecting if a sensor device is in contact with or in close proximity to the skin, the method comprising:
taking at least three measurements, including temperature, acceleration and capacitance, using different sensing elements; and
determining that the sensor device is in contact with or in close proximity to the skin of a user if at least two of the at least three measurements made by different sensing elements indicate that the sensor device is in contact with or in close proximity to the skin of a user.

13. The method of claim 12, wherein determining that the sensor device is in contact with or in close proximity to the skin of a user occurs when at least three of the at least three measurements made by different sensing elements indicate that the sensor device is in contact with or in close proximity to the skin of a user.

14. The method of claim 12 further comprising saving measurement data and wirelessly transmitting the measurement data to a remote unit.

15. The method of claim 14, wherein the sensor device has a unique identity and a predefined encryption key stored in the sensor device, wherein the method comprises the step of encrypting the measurement data using the encryption key before the measurement data is sent to the remote unit.

16. The method of claim 13 further comprising saving measurement data and wirelessly transmitting the measurement data to a remote unit.

17. The method of claim 16, wherein the sensor device has a unique identity and a predefined encryption key stored in the sensor device, wherein the method comprises the step of encrypting the measurement data using the encryption key before the measurement data is sent to the remote unit.

18. The method of claim 12 further comprising generating an alert when the sensor device is detached from a user.

19. The method of claim 12 further comprising detecting a body posture of a user during sleep using accelerometer measurements.

20. The method of claim 12 further comprising detecting heart rate and/or changes in skin redness and/or electrical conductivity.
